(19) United States
(12) Patent Application Publication
(10) Pub. No.: US 2015/0045435 A1
(43) Pub. Date: Feb. 12, 2015

(54) COMPOUNDS AND METHODS FOR TREATING DIABETES

(71) Applicant: Indiana University Research and Technology Corporation, Indianapolis, IN (US)

(72) Inventors: William L. Scott, Indianapolis, IN (US); Martin J. O'Donnell, Indianapolis, IN (US)

(21) Appl. No.: 14/452,910

(22) Filed: Aug. 6, 2014

Related U.S. Application Data
(60) Provisional application No. 61/862,764, filed on Aug. 6, 2013.

(51) Int. Cl. C07C 233/63

(52) U.S. Cl. CPC C07C 233/63
USPC 514/563; 562/450

(57) ABSTRACT

The invention described herein pertains to compounds, compositions, methods and uses thereof. The compounds described herein are in a class of glucose-lowering drugs useful for treating diabetes. Generally, compounds of the Formula I are described herein.

### CROSS-REFERENCE TO RELATED APPLICATIONS

This application claims the benefit, under 35 U.S.C. §119(e), of U.S. Provisional Application No. 61/862,764, which was filed Aug. 6, 2013, the entirety of each of the disclosures of which are hereby incorporated herein by reference.

### GOVERNMENT RIGHTS

This invention was made with government support under GM028193 awarded by the National Institutes of Health and 1140602 awarded by the National Science Foundation. The government has certain rights in the invention.

### TECHNICAL FIELD

The invention described herein pertains to compounds, compositions, methods and uses for treating diabetes.

### BACKGROUND AND SUMMARY OF THE INVENTION

Diabetes mellitus type 2 (type 2 diabetes), also referred to as non-insulin-dependent diabetes mellitus (NIDDM), is a metabolic disorder that is characterized by high blood glucose. In contrast to diabetes mellitus type 1 in which there is an absolute insulin deficiency due to destruction of islet cells in the pancreas, high blood glucose levels in type 2 diabetes may be due to insulin resistance and/or relative insulin deficiency. It has been reported that type 2 diabetes makes up about 90% of cases of diabetes with the other 10% due primarily to diabetes mellitus type 1 and gestational diabetes. Obesity is thought to be the primary cause of type 2 diabetes in people who are genetically predisposed to the disease.

It has also been reported that rates of diabetes have increased markedly over the last 50 years, and in parallel with obesity. As of 2010, there are reportedly approximately 285 million people with the disease compared to around 30 million in 1985. Long-term complications from high blood sugar can include heart disease, strokes, diabetic retinopathy where eyesight is affected, kidney failure which may require dialysis, and poor circulation of limbs leading to amputations.

Type 2 diabetes may be treated with lifestyle management, such as increasing exercise and dietary modification, and medication. Thus, there is a continuing need for additional treatment options.

Compounds for treating diabetes are described herein. Without being bound by theory, it is believed herein that the compounds lower blood glucose by stimulating the release of insulin from the pancreas. In addition, though without being bound by theory, it is believed herein that the compounds cause the closing of ATP-dependent potassium channels in the membrane of the β cells. This action depolarizes the β cells and causes voltage-gated calcium channels to open. The resulting calcium influx induces fusion of insulin-containing vesicles with the cell membrane, and insulin secretion occurs. The compounds described herein may have a mode of action similar to the meglitinide class of blood glucose-lowering drugs.

In one illustrative embodiment of the invention, described herein are compounds of the formula I

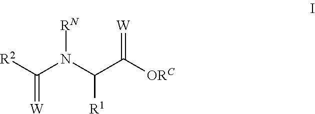

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted; and

R² is alkyl, alkenyl, alkynyl, cycloalkyl, cycloalkenyl, heteroalkyl, heteroalkenyl, cycloheteroalkyl, or cycloheteroalkenyl, each of which is optionally substituted;

wherein the compound is not of the formula

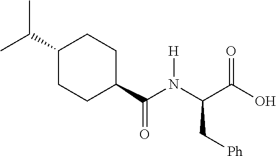 [SMILES: CC(C)[C@H]1CC[C@H](C(=O)N[C@H](Cc2ccccc2)C(=O)O)CC1]

or a pharmaceutically acceptable salt thereof.

In another illustrative embodiment of the invention, described herein are compounds of the formula I

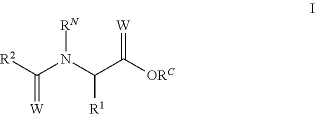

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted; and

R² is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted;

wherein the compound is not of the formula

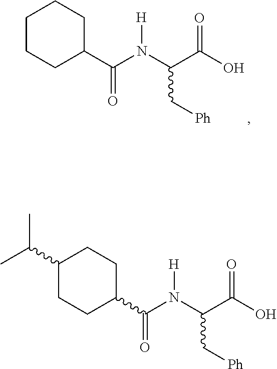 [SMILES: O=C(NC(Cc1ccccc1)C(=O)O)C1CCCCC1 ; CC(C)C1CCC(C(=O)NC(Cc2ccccc2)C(=O)O)CC1]

or a pharmaceutically acceptable salt thereof.

In another aspect of any one of the preceding embodiments, the compound wherein R² is cycloalkyl, cycloalkenyl, cycloheteroalkyl, or cycloheteroalkenyl, each of which is optionally substituted, or a pharmaceutically acceptable salt thereof. In another aspect of any one of the preceding embodiments, the compound wherein R² is cycloalkyl or cycloheteroalkyl, each of which is optionally substituted, or a pharmaceutically acceptable salt thereof. In another aspect of any one of the preceding embodiments, the compound wherein R² is optionally substituted cycloalkyl, or a pharmaceutically acceptable salt thereof. In another aspect of any one of the preceding embodiments, the compound wherein R² is cycloalkyl, or a pharmaceutically acceptable salt thereof. In another aspect of any one of the preceding embodiments, the compound wherein R² is selected from cyclopentyl, cyclohexyl, and cycloheptyl, each of which is optionally substituted, or a pharmaceutically acceptable salt thereof. In another aspect of any one of the preceding embodiments, the compound wherein R² is cyclopentyl, or a pharmaceutically acceptable salt thereof. In another aspect of any one of the preceding embodiments, the compound wherein R² is optionally substituted cyclopentyl, or a pharmaceutically acceptable salt thereof. In another aspect of any one of the preceding embodiments, the compound wherein R² is cyclopentyl optionally substituted with a C₁-C₆ alkyl, or a pharmaceutically acceptable salt thereof. In another aspect of any one of the preceding embodiments, the compound wherein R² is cyclopentyl, substituted with at least one C₁-C₆ alkyl, or a pharmaceutically acceptable salt thereof. In another aspect of any one of the preceding embodiments, the compound wherein R² is cyclohexyl, or a pharmaceutically acceptable salt thereof. In another aspect of any one of the preceding embodiments, the compound wherein R² is optionally substituted cyclohexyl, or a pharmaceutically acceptable salt thereof. In another aspect of any one of the preceding embodiments, the compound wherein R² is cyclohexyl optionally substituted with a C₁-C₆ alkyl, or a pharmaceutically acceptable salt thereof. In another aspect of any one of the preceding embodiments, the compound wherein R² is cyclohexyl, substituted with at least one C₁-C₆ alkyl, or a pharmaceutically acceptable salt thereof. In another aspect of any one of the preceding embodiments, the compound wherein R² is cycloheptyl, or a pharmaceutically acceptable salt thereof. In another aspect of any one of the preceding embodiments, the compound wherein R² is optionally substituted cycloheptyl, or a pharmaceutically acceptable salt thereof. In another aspect of any one of the preceding embodiments, the compound wherein R² is cycloheptyl optionally substituted with a C₁-C₆ alkyl, or a pharmaceutically acceptable salt thereof. In another aspect of any one of the preceding embodiments, the compound wherein R² is cycloheptyl, substituted with at least one C₁-C₆ alkyl, or a pharmaceutically acceptable salt thereof.

In another aspect of any one of the preceding embodiments, the compound wherein W is O, or a pharmaceutically acceptable salt thereof. In another aspect of any one of the preceding embodiments, the compound wherein R^{N} is H, or a pharmaceutically acceptable salt thereof. In another aspect of any one of the preceding embodiments, the compound wherein R^{C} is H, or a pharmaceutically acceptable salt thereof. In another aspect of any one of the preceding embodiments, the compound wherein R¹ is optionally substituted arylalkyl, or a pharmaceutically acceptable salt thereof. In another aspect of any one of the preceding embodiments, the compound wherein R¹ is optionally substituted benzyl, or a pharmaceutically acceptable salt thereof. In another aspect of any one of the preceding embodiments, the compound wherein R¹ is benzyl, or a pharmaceutically acceptable salt thereof. In another aspect of any one of the preceding embodiments, the compound wherein R¹ is substituted benzyl, or a pharmaceutically acceptable salt thereof. In another aspect of any one of the preceding embodiments, the compound wherein R¹ is benzyl optionally substituted with one or more halogen atom, or a pharmaceutically acceptable salt thereof. In another aspect of any one of the preceding embodiments, the compound wherein R¹ is benzyl having one or more hydrogen atoms attached thereto substituted by a fluorine atom, or a pharmaceutically acceptable salt thereof. In another aspect of any one of the preceding embodiments, the compound wherein R¹ is fluorobenzyl, or a pharmaceutically acceptable salt thereof. In another aspect of any one of the preceding embodiments, the compound wherein R¹ is 2-fluorobenzyl, or a pharmaceutically acceptable salt thereof. In another aspect of any one of the preceding embodiments, the compound wherein R¹ is 3-fluorobenzyl, or a pharmaceutically acceptable salt thereof. In another aspect of any one of the preceding embodiments, the compound wherein R¹ is 4-fluorobenzyl, or a pharmaceutically acceptable salt thereof. In another aspect of any one of the preceding embodiments, the compound wherein R¹ is benzyl having one or more hydrogen atoms attached thereto substituted by a chlorine atom, or a pharmaceutically acceptable salt thereof. In another aspect of any one of the preceding embodiments, the compound wherein R¹ is chlorobenzyl, or a pharmaceutically acceptable salt thereof. In another aspect of any one of the preceding embodiments, the compound wherein R¹ is 2-chlorobenzyl, or a pharmaceutically acceptable salt thereof. In another aspect of any one of the preceding embodiments, the compound wherein R¹ is 3-chlorobenzyl, or a pharmaceutically acceptable salt thereof. In another aspect of any one of the preceding embodiments, the compound wherein R¹ is 4-chlorobenzyl, or a pharmaceutically acceptable salt thereof. In another aspect of any one of the preceding embodiments, the compound wherein R¹ is benzyl having one or more hydrogen atoms attached thereto substituted by a bromine atom, or a pharmaceutically acceptable salt thereof. In another aspect of any one of the preceding embodiments, the compound wherein R¹ is bromobenzyl, or a pharmaceutically acceptable salt thereof. In another aspect of any one of the preceding embodiments, the compound wherein R¹ is 2-bromobenzyl, or a pharmaceutically acceptable salt thereof. In another aspect of any one of the preceding embodiments, the compound wherein R¹ is 3-bromobenzyl, or a pharmaceutically acceptable salt thereof. In another aspect of any one of the preceding embodiments, the compound wherein R¹ is 4-bromobenzyl, or a pharmaceutically acceptable salt thereof.

In another illustrative embodiment of the invention, described herein are compounds of the formula II

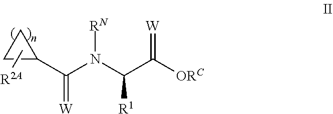

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted;

R^{2A} is hydrogen, alkyl, alkenyl, alkynyl, cycloalkyl, cycloalkenyl, heteroalkyl, heteroalkenyl, cycloheteroalkyl, or cycloheteroalkenyl, aryl, arylalkyl, arylheteroalkyl, heteroaryl, heteroarylalkyl, or heteroarylheteroalkyl, each of which is optionally substituted; and

n is 1, 2, 3, 4. 5, or 6,

wherein the compound is not of the formula

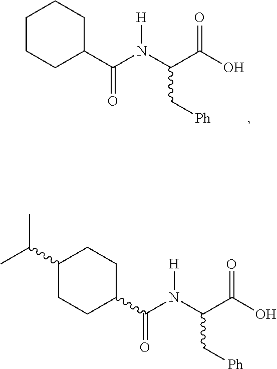 [SMILES: O=C(NC(Cc1ccccc1)C(=O)O)C1CCCCC1 ; CC(C)C1CCC(C(=O)NC(Cc2ccccc2)C(=O)O)CC1]

or a pharmaceutically acceptable salt thereof.

In another illustrative embodiment of the invention, described herein are compounds of the formula III

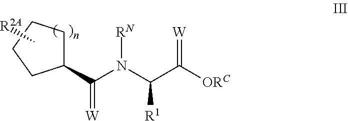

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted;

R^{2A} is alkyl, alkenyl, alkynyl, cycloalkyl, cycloalkenyl, heteroalkyl, heteroalkenyl, cycloheteroalkyl, or cycloheteroalkenyl, aryl, arylalkyl, arylheteroalkyl, heteroaryl, heteroarylalkyl, or heteroarylheteroalkyl, each of which is optionally substituted; and

n is 1, 2, 3, or 4,

wherein the compound is not of the formula

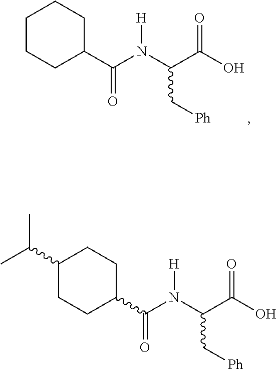 [SMILES: O=C(NC(Cc1ccccc1)C(=O)O)C1CCCCC1 ; CC(C)C1CCC(C(=O)NC(Cc2ccccc2)C(=O)O)CC1]

or a pharmaceutically acceptable salt thereof.

In another aspect of any one of the preceding embodiments, the compound wherein R^{2A} is C₁-C₆ alkyl, or a pharmaceutically acceptable salt thereof. In another aspect of any one of the preceding embodiments, the compound wherein W is O, or a pharmaceutically acceptable salt thereof. In another aspect of any one of the preceding embodiments, the compound wherein R^{N} is H, or a pharmaceutically acceptable salt thereof. In another aspect of any one of the preceding embodiments, the compound wherein R^{C} is H, or a pharmaceutically acceptable salt thereof. In another aspect of any one of the preceding embodiments, the compound wherein R¹ is optionally substituted arylalkyl, or a pharmaceutically acceptable salt thereof. In another aspect of any one of the preceding embodiments, the compound wherein R¹ is optionally substituted benzyl, or a pharmaceutically acceptable salt thereof. In another aspect of any one of the preceding embodiments, the compound wherein R¹ is benzyl, or a pharmaceutically acceptable salt thereof. In another aspect of any one of the preceding embodiments, the compound wherein R¹ is substituted benzyl, or a pharmaceutically acceptable salt thereof. In another aspect of any one of the preceding embodiments, the compound wherein R¹ is benzyl optionally substituted with one or more halogen atom, or a pharmaceutically acceptable salt thereof. In another aspect of any one of the preceding embodiments, the compound wherein R¹ is benzyl having one or more hydrogen atoms attached thereto substituted by a fluorine atom, or a pharmaceutically acceptable salt thereof. In another aspect of any one of the preceding embodiments, the compound wherein R¹ is fluorobenzyl, or a pharmaceutically acceptable salt thereof. In another aspect of any one of the preceding embodiments, the compound wherein R¹ is 2-fluorobenzyl, or a pharmaceutically acceptable salt thereof. In another aspect of any one of the preceding embodiments, the compound wherein R¹ is 3-fluorobenzyl, or a pharmaceutically acceptable salt thereof. In another aspect of any one of the preceding embodiments, the compound wherein R¹ is 4-fluorobenzyl, or a pharmaceutically acceptable salt thereof. In another aspect of any one of the preceding embodiments, the compound wherein R¹ is benzyl having one or more hydrogen atoms attached thereto substituted by a chlorine atom, or a pharmaceutically acceptable salt thereof. In another aspect of any one of the preceding embodiments, the compound wherein R¹ is chlorobenzyl, or a pharmaceutically acceptable salt thereof. In another aspect of any one of the preceding embodiments, the compound wherein R¹ is 2-chlorobenzyl, or a pharmaceutically acceptable salt thereof. In another aspect of any one of the preceding embodiments, the compound wherein R¹ is 3-chlorobenzyl, or a pharmaceutically acceptable salt thereof. In another aspect of any one of the preceding embodiments, the compound wherein R¹ is 4-chlorobenzyl, or a pharmaceutically acceptable salt thereof. In another aspect of any one of the preceding embodiments, the compound wherein R¹ is benzyl having one or more hydrogen atoms attached thereto substituted by a bromine atom, or a pharmaceutically acceptable salt thereof. In another aspect of any one of the preceding embodiments, the compound wherein R¹ is bromobenzyl, or a pharmaceutically acceptable salt thereof. In another aspect of any one of the preceding embodiments, the compound wherein R¹ is 2-bromobenzyl, or a pharmaceutically acceptable salt thereof. In another aspect of any one of the preceding embodiments, the compound wherein R¹ is 3-bromobenzyl, or a pharmaceutically acceptable salt thereof. In another aspect of any one of the preceding embodiments, the compound wherein R¹ is 4-bromobenzyl, or a pharmaceutically acceptable salt thereof.

In another illustrative embodiment of the invention, described herein are compounds selected from the group consisting of

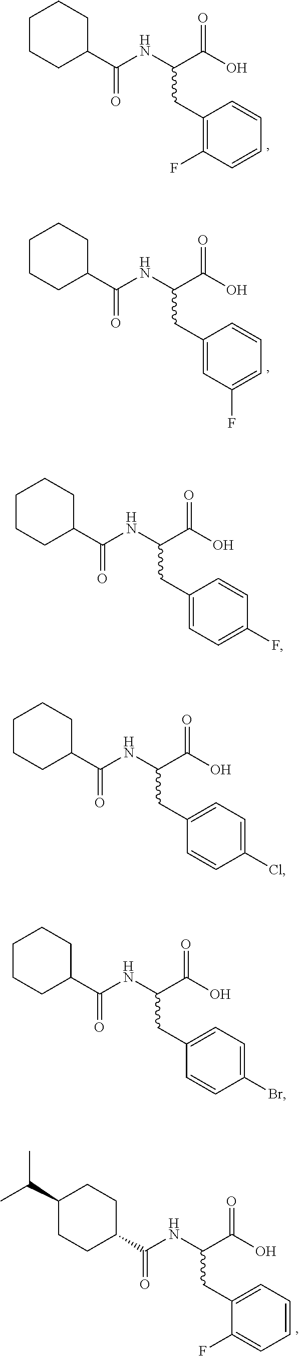 [SMILES: O=C(NC(Cc1ccccc1F)C(=O)O)C1CCCCC1 ; O=C(NC(Cc1cccc(F)c1)C(=O)O)C1CCCCC1 ; O=C(NC(Cc1ccc(F)cc1)C(=O)O)C1CCCCC1 ; O=C(NC(Cc1ccc(Cl)cc1)C(=O)O)C1CCCCC1 ; O=C(NC(Cc1ccc(Br)cc1)C(=O)O)C1CCCCC1 ; CC(C)[C@H]1CC[C@H](C(=O)NC(Cc2ccccc2F)C(=O)O)CC1]

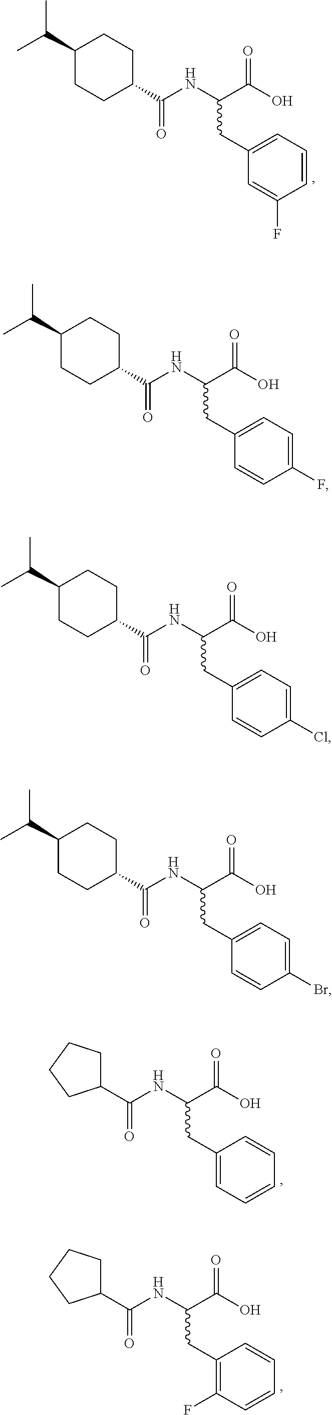 [SMILES: CC(C)[C@H]1CC[C@H](C(=O)NC(Cc2cccc(F)c2)C(=O)O)CC1 ; CC(C)[C@H]1CC[C@H](C(=O)NC(Cc2ccc(F)cc2)C(=O)O)CC1 ; CC(C)[C@H]1CC[C@H](C(=O)NC(Cc2ccc(Cl)cc2)C(=O)O)CC1 ; CC(C)[C@H]1CC[C@H](C(=O)NC(Cc2ccc(Br)cc2)C(=O)O)CC1 ; O=C(NC(Cc1ccccc1)C(=O)O)C1CCCC1 ; O=C(NC(Cc1ccccc1F)C(=O)O)C1CCCC1]

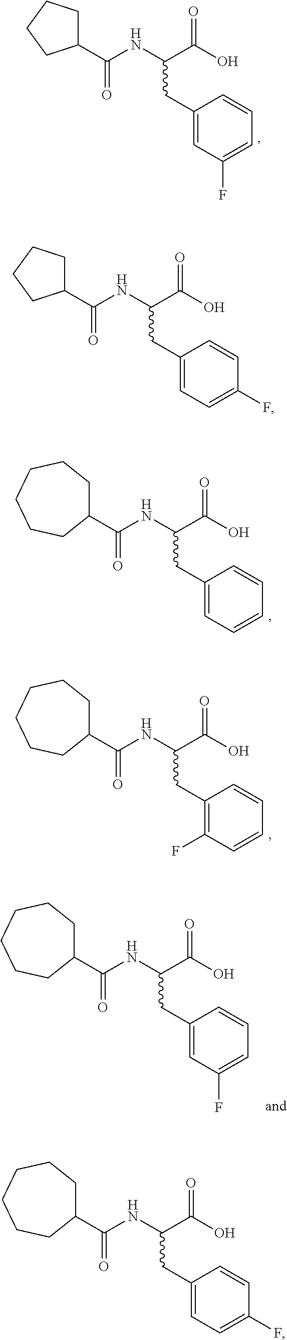 [SMILES: O=C(NC(Cc1cccc(F)c1)C(=O)O)C1CCCC1 ; O=C(NC(Cc1ccc(F)cc1)C(=O)O)C1CCCC1 ; O=C(NC(Cc1ccccc1)C(=O)O)C1CCCCCC1 ; O=C(NC(Cc1ccccc1F)C(=O)O)C1CCCCCC1 ; O=C(NC(Cc1cccc(F)c1)C(=O)O)C1CCCCCC1 ; O=C(NC(Cc1ccc(F)cc1)C(=O)O)C1CCCCCC1]

or a pharmaceutically acceptable salt thereof. Various other embodiments of the invention are described as follows. It will be understood that the following embodiments may be combined with other embodiments described herein. In one embodiment, pharmaceutical compositions containing one or more of the compounds are described herein. In another embodiment, pharmaceutical compositions containing one or more of the compounds described herein, and one or more carriers, diluents, or excipients. In one aspect, the compositions include a therapeutically effective amount of the one or more compounds for treating a patient with a disease responsive to increased insulin production, such as diabetes. It is to be understood that the compositions may include other component and/or ingredients, including, but not limited to, other therapeutically active compounds, and/or one or more carriers, diluents, excipients, and the like. In another embodiment, methods for using the compounds and pharmaceutical compositions for treating patients with a disease responsive to increased insulin production, such as diabetes are also described herein. In one aspect, the methods include the step of administering one or more of the compounds and/or compositions described herein to a patient with a disease responsive to increased insulin production, such as diabetes. In one embodiment, described herein is a method for treating a disease responsive to increased insulin production in a mammal in need of such treatment, the method comprising the step of administering to the mammal a therapeutically effective amount of a compound of claim 1. In another embodiment, the mammal is a human. In another embodiment, the disease responsive to increased insulin production is type 2 diabetes. In another aspect, the methods include administering a therapeutically effective amount of the one or more compounds and/or compositions described herein for treating patients with a disease responsive to increased insulin production, such as diabetes. In another embodiment, uses of the compounds and compositions in the manufacture of a medicament for treating patients with a disease responsive to increased insulin production, such as diabetes are also described herein. In one aspect, the medicaments include a therapeutically effective amount of the one or more compounds and/or compositions for treating a patient with a disease responsive to increased insulin production, such as diabetes.

It is appreciated herein that the compounds described herein may be used alone or in combination with other compounds useful for treating a disease responsive to increased insulin production, such as diabetes, including those compounds that may be therapeutically effective by the same or different modes of action. In addition, it is appreciated herein that the compounds described herein may be used in combination with other compounds that are administered to treat other symptoms of a disease responsive to increased insulin production, such as diabetes.

### BRIEF DESCRIPTION OF THE DRAWINGS

FIG. 1 shows results of certain of the compounds described herein in the glucose stimulated insulin secretion assay. Results are described in μLU/mg of insulin/cell protein for the test compounds and reference standards.

FIG. 2 shows results of certain of the compounds described herein in the glucose stimulated insulin secretion assay. Results are described in μLU/mg of insulin/cell protein for the test compounds and reference standards.

FIG. 3 shows results of certain of the compounds described herein in the glucose stimulated insulin secretion assay. Results are described in μLU/mg of insulin/cell protein for the test compounds and reference standards.

FIG. 4 shows results of certain of the compounds described herein in the glucose stimulated insulin secretion assay. Results are described in μLU/mg of insulin/cell protein for the test compounds and reference standards.

FIG. 5 shows results of certain of the compounds described herein in the glucose stimulated insulin secretion assay. Results are described in μLU/mg of insulin/cell protein for the test compounds and reference standards.

FIG. 6 shows results of certain of the compounds described herein in the glucose stimulated insulin secretion assay. Results are described in μLU/mg of insulin/cell protein for the test compounds and reference standards.

FIG. 7 shows results of certain of the compounds described herein in the glucose stimulated insulin secretion assay. Results are described in μLU/mg of insulin/cell protein for the test compounds and reference standards.

FIG. 8 shows results of certain of the compounds described herein in the glucose stimulated insulin secretion assay. Results are described in μLU/mg of insulin/cell protein for the test compounds and reference standards.

FIG. 9 shows results of certain of the compounds described herein in the glucose stimulated insulin secretion assay. Results are described in μLU/mg of insulin/cell protein for the test compounds and reference standards.

### DETAILED DESCRIPTION

Several illustrative embodiments of the invention are described by the following enumerated clauses:

1. A compound of the formula I

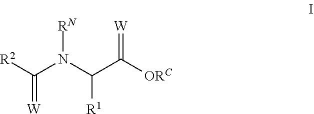

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted; and

R² is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted;

wherein the compound is not of the formula

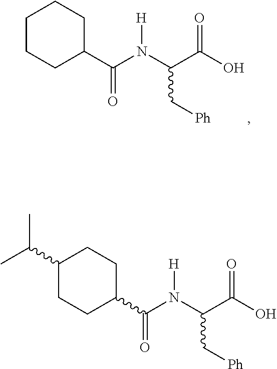 [SMILES: O=C(NC(Cc1ccccc1)C(=O)O)C1CCCCC1 ; CC(C)C1CCC(C(=O)NC(Cc2ccccc2)C(=O)O)CC1]

or a pharmaceutically acceptable salt thereof.

2. The compound of clause 1 of the formula

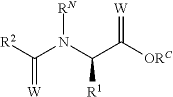

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted; and

R² is cycloalkyl, cycloalkenyl, cycloheteroalkyl, cycloheteroalkenyl, aryl, heteroaryl, arylalkyl, or heteroarylalkyl, each of which is optionally substituted.

3. The compound of any one of clauses 1 to 2 wherein R² is cycloalkyl, cycloalkenyl, cycloheteroalkyl, or cycloheteroalkenyl, each of which is optionally substituted.

4. The compound of any one of clauses 1 to 2 wherein R² is cycloalkyl or cycloheteroalkyl, each of which is optionally substituted.

5. The compound of any one of clauses 1 to 2 wherein R² is optionally substituted cycloalkyl.

6. The compound of any one of clauses 1 to 2 wherein R² is cycloalkyl.

7. The compound of clause 1 of the formula II

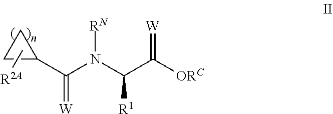

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted;

R^{2A} is hydrogen, alkyl, alkenyl, alkynyl, cycloalkyl, cycloalkenyl, heteroalkyl, heteroalkenyl, cycloheteroalkyl, or cycloheteroalkenyl, aryl, arylalkyl, arylheteroalkyl, heteroaryl, heteroarylalkyl, or heteroarylheteroalkyl, each of which is optionally substituted; and

n is 1, 2, 3, 4. 5, or 6.

8. The compound of clause 7 wherein R^{2A} is alkyl, alkenyl, alkynyl, cycloalkyl, cycloalkenyl, heteroalkyl, heteroalkenyl, cycloheteroalkyl, or cycloheteroalkenyl, aryl, arylalkyl, arylheteroalkyl, heteroaryl, heteroarylalkyl, or heteroarylheteroalkyl, each of which is optionally substituted.

9. The compound of clause 7 wherein R^{2A} is alkyl.

10. The compound of clause 1 of the formula III

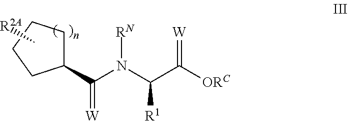

or a pharmaceutically acceptable salt thereof, wherein:

W is independently O or S;

R^{N} is H, or a amide prodrug forming group;

R^{C} is H, or a carboxylic acid prodrug forming group;

R¹ is arylalkyl or heteroarylalkyl, each of which is optionally substituted;

R^{2A} is alkyl, alkenyl, alkynyl, cycloalkyl, cycloalkenyl, heteroalkyl, heteroalkenyl, cycloheteroalkyl, or cycloheteroalkenyl, aryl, arylalkyl, arylheteroalkyl, heteroaryl, heteroarylalkyl, or heteroarylheteroalkyl, each of which is optionally substituted; and

n is 1, 2, 3, or 4.

11. The compound of clause 10 wherein R^{2A} is alkyl.

12. The compound of any one of clauses 1 to 12 wherein each W is O.

13. The compound of any one of clauses 1 to 13 wherein R^{N} is H.

14. The compound of any one of clauses 1 to 14 wherein R^{C} is H.

15. The compound of any one of clauses 1 to 15 wherein R¹ is optionally substituted arylalkyl.

16. The compound of any one of clauses 1 to 15 wherein R¹ is optionally substituted benzyl.

17. The compound of any one of clauses 1 to 15 wherein R¹ is benzyl.

18. The compound of any one of clauses 1 to 15 wherein R¹ is substituted benzyl.

19. The compound of any one of clauses 1 to 15 wherein R¹ is benzyl substituted with one or more fluoro.

20. A pharmaceutical composition comprising one or more compounds of any one of clauses 1 to 21.

21. A unit dose or unit dosage form composition comprising a therapeutically effective amount of one or more compounds of any one of clauses 1 to 21 for treating a disease responsive to increased insulin production in a host animal.

22. The composition or unit dose or unit dosage form of clause 20 or 21 further comprising one or more carriers, diluents, or excipients, or a combination thereof.

23. A method for treating a disease responsive to increased insulin production in a host animal, the method comprising the step of administering to the host animal a composition comprising a therapeutically effective amount of one or more compounds of any one of clauses 1 to 19; or a pharmaceutical composition or unit dose thereof, and optionally further comprising one or more carriers, diluents, or excipients, or a combination thereof.

24. Use of one or more compounds of any one of clauses 1 to 19; or a pharmaceutical composition or unit dose thereof, and optionally further comprising one or more carriers, diluents, or excipients, or a combination thereof in the manufacture of a medicament for treating a disease responsive to increased insulin production in a host animal.

25. The unit dose, method, or use of any one of clauses 21 to 24 wherein the host animal is a human.

26. The unit dose, method, or use of any one of clauses 21 to 25 wherein the disease is non-insulin-dependent diabetes mellitus.

In each of the foregoing and following embodiments, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non crystalline and/or amorphous forms of the compounds.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers. As used herein, the symbol “ [symbol] ” denotes a covalent bond connection where stereochemistry is possible, but a mixture of stereoisomers is described. For example, in certain embodiments, the carbon atom to which “ [symbol] ” is attached can be a chiral center having enantiomers denoted R and S according to the fundamental rules of organic chemistry, but use of the symbol “ [symbol] ” denotes that a mixture of enantiomers is described. More particularly, the compounds described in the examples showing the symbol “ [symbol] 37 ” connecting the benzyl group to the molecule exist as a mixture of possible stereoisomers.

Similarly, the compounds described herein may be include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. It is to be further understood that in certain embodiments alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior. Illustrative alkyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “arylalkyl” includes monocyclic and polycyclic aromatic carbocyclic groups where at least one hydrogen atom attached thereto is substituted by an alkyl moiety, each of which may be optionally substituted. It will be understood that arylalkyl groups can be attached to other functional groups or moieties at any position including, without limitation at an atom on the alkyl moiety (e.g. benzyl) or at an atom on the monocyclic and polycyclic aromatic carbocyclic group (e.g. o-methylphenyl). Illustrative arylalkyl groups described herein include, but are not limited to, benzyl, o-methylphenyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)O₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{x}, where x is an integer from 0-6 and Z^{x} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{x} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

### EXAMPLES

General Procedure I

The compounds described herein may be prepared according to the processes described herein and/or conventional process. Illustratively, the compounds described herein may be prepared according to the following process:

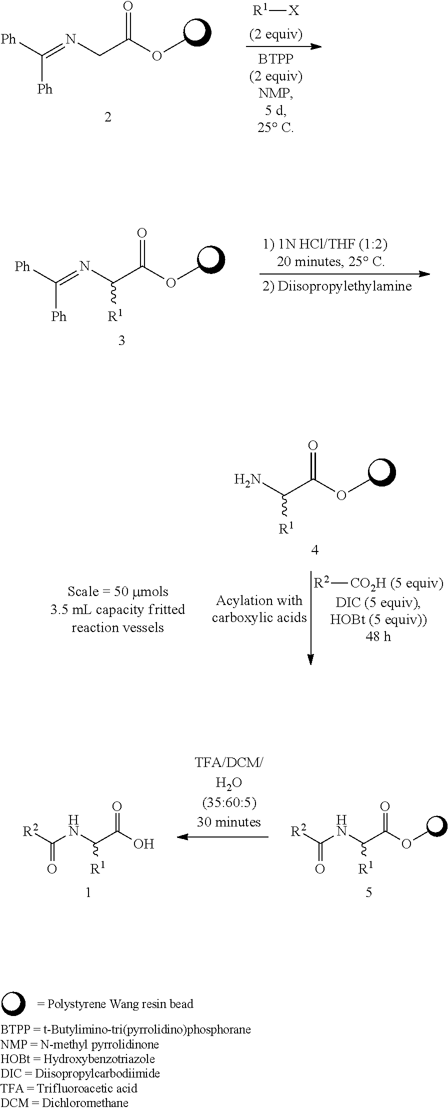

where it is to be understood that additional compounds described herein are prepared by the selection of the corresponding starting compounds R¹—X, R²—CO₂H, and the like.

The following examples further illustrate specific embodiments of the invention; however, the following illustrative examples should not be interpreted in any way to limit the invention.

### Example A1

### Compound A1 was Prepared According to General Procedure I Described Above

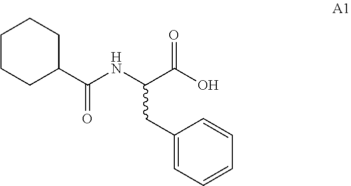 [SMILES: O=C(NC(Cc1ccccc1)C(=O)O)C1CCCCC1]

¹H NMR (500 MHz, CDCl₃) δ 1.17-1.39 (m, 5H), 1.65-1.66 (m, 1H), 1.74-1.82 (m, 4H), 2.08 (tt, J=8.3, 11.7, 15, 1H), 3.14 (dd, J=6.6, 14.2, 1H), 3.26 (dd, J=5.6, 14.2, 1H), 4.83 (q, J=6.6, 12.5, 1H), 5.86 (d, J=7.1, 1H), 7.15-7.17 (m, 2H), 7.27-7.33 (m, 3H) ¹³C NMR (125 MHz, CDCl₃): δ 25.49, 25.58, 25.60, 29.23, 29.52, 36.97, 45.1, 53.2, 127.3, 128.7, 129.3, 135.6, 173.8, 176.9

HRMS: calcd for C₁₆H₂₁NO₃, (M+H)⁺276.1594, found 276.1591

### Example A2

### Compound A2 was Prepared According to General Procedure I Described Above

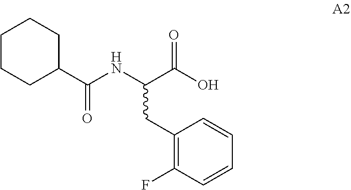 [SMILES: O=C(NC(Cc1ccccc1F)C(=O)O)C1CCCCC1]

¹H NMR (500 MHz, CDCl₃) δ 1.19-1.36 (m, 5H), 1.64-1.66 (m, 1H), 1.74-1.82 (m, 4H), 2.09 (tt, J=3.4, 14.7, 1H), 3.16 (dd, J=7.5, 14.2, 1H), 3.30 (dd, J=5.3, 14.2, 1H), 4.82 (q, J=7.5, 1H), 6.06 (d, J=7.4), 7.02-7.10 (m, 2H), 7.18-7.25 (m, 2H)

¹³C NMR (125 MHz, CDCl₃): δ 25.51, 25.59, 25.61, 29.2, 29.4, 30.7, 45.0, 52.8, 115.4 (d, J=21.3), 122.9 (d, J=16.3), 124.4 (d, J=3), 129.2 (d, J=7.5), 131.8 (d, J=5), 161.3 (d, J¹=244), 173.9, 177.1

HRMS: calcd for C₁₆H₂₀FNO₃, (M+H)⁺294.1500, found 294.1495

### Example A3

### Compound A3 was Prepared According to General Procedure I Described Above

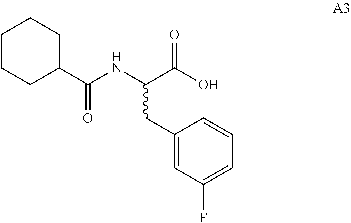 [SMILES: O=C(NC(Cc1cccc(F)c1)C(=O)O)C1CCCCC1]

¹H NMR (500 MHz, CDCl₃) δ 1.20-1.25 (m, 3H), 1.33-1.41 (m, 2H), 1.66-1.67 (m, 1 H), 1.76-1.83 (m, 4H), 2.11 (tt, J=3.4, 11.7, 1H), 3.13 (dd, J=6.2, 14.1, 1H), 3.26 (dd, J=5.7, 14.1, 1H), 4.85 (q, J=6.1, 13.2, 1H), 5.95 (d, J=7.2, 1H), 6.86-6.88 (m, 1H), 6.93-6.98 (m, 2H), 7.25-7.29 (m, 1H)

¹³C NMR (125 MHz, CDCl₃): δ 25.50, 25.58, 25.59, 29.3, 29.5, 36.9, 45.1, 52.9, 114.2 (d, J=20.8), 116.3 (d, J=21.0), 125.1 (d, J=2.7), 130.1 (d, J=8.2), 138.2 (d, J=7.3), 162.5 (d, J¹=175), 174.0, 176.8

HRMS: calcd for C₁₆H₂₀FNO₃, (M+H)⁺294.1500, found 294.1506

### Example A4

### Compound A4 was Prepared According to General Procuedure I Described Above

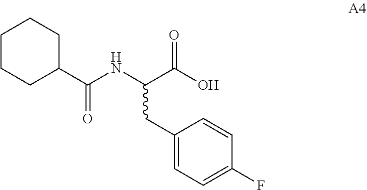 [SMILES: O=C(NC(Cc1ccc(F)cc1)C(=O)O)C1CCCCC1]

¹H NMR (500 MHz, CDCl₃) δ 1.20-1.26 (m, 4H), 1.35-1.38 (m, 2H), 1.66-1.68 (m, 1H), 1.76-1.83 (m, 4H), 2.12 (tt, J=8.3, 11.7, 1H), 3.10 (dd, J=6.1, 14.1, 1H), 3.22 (dd, J=5.7, 14.2, 1H), 4.80 (q, J=5.9, 12.0, 1H), 5.99 (d, J=7.4, 1H), 6.97-7.00 (m, 2H), 7.10-7.13 (m, 2H)

¹³C NMR (125 MHz, CDCl₃): δ 25.5, 25.6, 29.3, 29.6, 36.4, 45.1, 53.0, 115.5 (d, J=21.0), 130.9 (d, J=7.8), 131.4 (d, J=3.4), 162.1 (d, J¹=246), 174.4, 176.8

HRMS: calcd for C₁₆H₂₀FNO₃, (M+H)⁺294.1500, found 294.1500

### Example A5

### Compound A5 was Prepared According to General Procuedure I Described Above

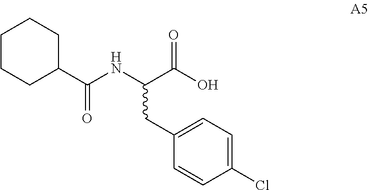 [SMILES: O=C(NC(Cc1ccc(Cl)cc1)C(=O)O)C1CCCCC1]

¹H NMR (500.13 MHz, CDCl₃): δ 1.20 (m), 1.76 (m), 2.11 (m), 3.11 (q), 3.23 (q), 4.83 (q), 5.91 (d), 7.26 (m, 4H)

¹³C NMR: δ 25.5, 25.6, 29.3, 29.6, 36.5, 45.1, 52.9, 128.8, 130.7, 133.2, 134.2, 173.9, 176.8

LC/MS calcd for C₁₆H₂₀ClNO₃ (M+H)⁺309.39, found 310.0.

### Example A6

### Compound A6 was Prepared According to General Procedure I Described Above

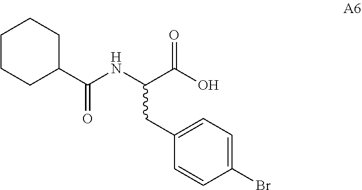 [SMILES: O=C(NC(Cc1ccc(Br)cc1)C(=O)O)C1CCCCC1]

¹H NMR (500.13 MHz, CDCl₃): δ 1.27 (m), 1.76 (m), 2.10, (m), 3.06 (q), 3.18 (m), 4.79 (m), 5.97 (d), 7.19 (m)

¹³C NMR: δ 25.5, 25.6, 29.3, 29.6, 36.6, 45.1, 52.9, 121.3, 131.1, 131.7, 134.7, 173.9, 176.7

LC/MS calcd for C₁₆H₂₀BrNO₃ (M+H)⁺354.24, found 354.0.

### Example A7

### Compound A7 was Prepared According to General Procedure I Described Above

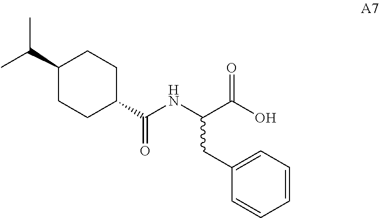 [SMILES: CC(C)[C@H]1CC[C@H](C(=O)NC(Cc2ccccc2)C(=O)O)CC1]

¹H NMR (500.13 MHz, CDCl₃): δ 0.95 (m), 1.37 (m), 1.91 (m), 3.13 (q), 3.25 (q), 4.86 (s), 6.01 (d), 7.21 (m).

¹³C NMR: δ 19.7, 28.8, 28.9, 29.4, 29.6, 32.8, 36.7, 43.1, 45.3, 126.8, 128.8, 129.5, 136.7, 177.0.

LC/MS calcd for C₁₉H₂₇NO₃ (M+H)⁺318.42, found 318.1.

### Example A8

### Compound A8 was Prepared According to General Procedure I Described Above

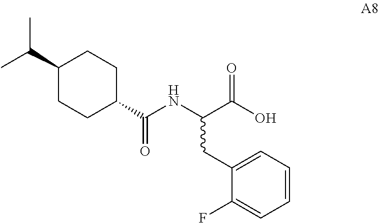 [SMILES: CC(C)[C@H]1CC[C@H](C(=O)NC(Cc2ccccc2F)C(=O)O)CC1]

¹H NMR (500.13 MHz, CDCl₃): δ 0.96 (m), 1.35 (m), 1.87 (m), 3.15 (q), 3.31 (q),4.84 (q), 6.13 (d), 7.14 (m)

¹³C NMR: δ 28.8, 28.9, 29.4, 29.7, 32.7, 36.5, 43.2, 45.4, 53.0, 115.4, 115.6, 123.5, 123.6, 124.8, 129.5, 131.8, 160.1, 161.8, 173.8, 176.9

LC/MS calcd for C₁₉H₂₆FNO₃ (M+H)⁺336.41, found 336.2

### Example A9

### Compound A9 was Prepared According to General Procedure I Described Above

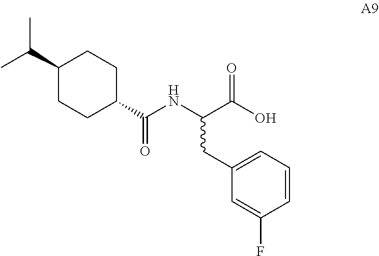 [SMILES: CC(C)[C@H]1CC[C@H](C(=O)NC(Cc2cccc(F)c2)C(=O)O)CC1]

¹H NMR (500.13 MHz, CDCl₃): δ 0.97 (m), 1.38 (m), 1.91 (m), 3.12 (q), 3.26 (q), 4.85 (m), 6.01 (d), 6.93 (m), 7.26 (m)

¹³C NMR: δ 19.7, 28.8, 28.9, 29.4, 29.7, 32.7, 36.9, 43.2, 45.4, 114.1, 114.3, 116.3, 116.4, 125.1, 130.0, 130.1, 138.2, 138.3, 161.9, 163.8, 173.9, 176.9

LC/MS calcd for C₁₉H₂₆FNO₃ (M+H)⁺336.41, found 336.2.

Example A10: Compound A10 was Prepared According to General Procedure I Described Above.

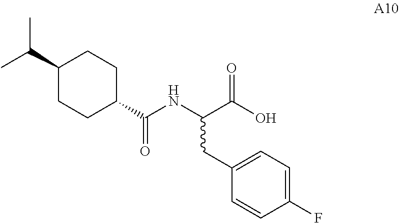 [SMILES: CC(C)[C@H]1CC[C@H](C(=O)NC(Cc2ccc(F)cc2)C(=O)O)CC1]

¹H NMR (500.13 MHz, CDCl₃): δ 0.98 (m), 1.37 (m), 1.94 (m), 3.10 (q), 3.23 (q), 4.82 (q), 5.95 (d), 7.05 (m)

¹³C NMR: δ 19.7, 28.8, 28.9, 29.5, 29.7, 32.7, 36.4, 43.2, 45.4, 53.1, 115.4, 115.6, 130.8, 130.9, 131.4, 161.1, 163.0, 176.9

LC/MS calcd for C₁₉H₂₆FNO₃ (M+H)⁺336.41, found 336.2.

Example A11

### Compound A11 was Prepared According to General Procedure I Described Above

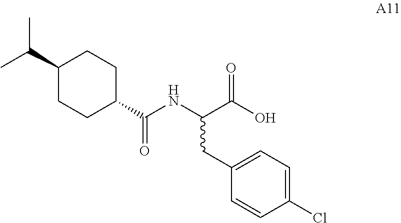 [SMILES: CC(C)[C@H]1CC[C@H](C(=O)NC(Cc2ccc(Cl)cc2)C(=O)O)CC1]

¹H NMR (500.13 MHz, CDCl₃): δ 0.98 (m), 1.37 (m), 1.97 (m), 3.10 (q), 3.22 (q), 4.82 (q), 5.91 (d), 7.09 (d), 7.26 (d)

¹³C NMR: δ 19.7, 28.8, 28.9, 29.4, 29.7, 32.7, 36.5, 43.2, 45.4, 53.0, 128.8, 130.7, 133.2, 134.2, 173.8, 176.8

LC/MS calcd for C₁₉H₂₆ClNO₃ (M+H)⁺351.87, found 352.1.

### Example A12

### Compound A12 was Prepared According to General Procedure I Described Above

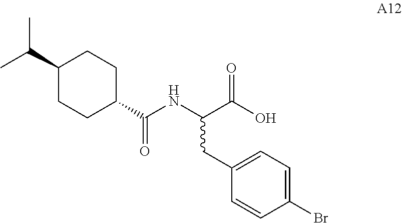 [SMILES: CC(C)[C@H]1CC[C@H](C(=O)NC(Cc2ccc(Br)cc2)C(=O)O)CC1]

¹H NMR (500.13 MHz, CDCl₃): δ .093 (m), 1.38 (m), 1.94 (m), 3.09 (q), 3.21 (q), 4.81 (q), 5.91 (d), 7.03 (d), 7.28 (d), 7.43 (d)

¹³C NMR: δ 19.7, 28.8, 28.9, 29.5, 29.7, 32.7, 36.6, 43.2, 45.4, 52.9, 121.3, 131.1, 131.8, 134.7, 173.8, 176.8

LC/MS calcd for C₁₉H₂₆ClNO₃ (M+H)⁺396.32, found 396.1.

### Example B1

### Compound B1 was Prepared According to General Procedure I Described Above.

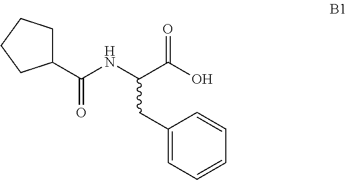 [SMILES: O=C(NC(Cc1ccccc1)C(=O)O)C1CCCC1]

¹H NMR (500 MHz, CDCl₃) δ 1.53-1.56 (m, 2H), 1.66-1.75 (m, 4H), 1.80-1.83 (m, 2H), 2.52 (p, J=7.9, 15.8, 1H), 3.13 (dd, J=6.5, 14.1, 1H), 3.25 (dd, J=5.6, 14.1, 1H), 4.86 (q, J=6.4, 13.0, 1H), 5.91 (d, J=7.3, 1H), 7.15-7.17 (m, 2H), 7.24-7.32 (m, 3H)

¹³C NMR (125 MHz, CDCl₃): δ 25.8, 25.9, 30.0, 30.5, 37.1, 45.6, 53.5, 127.3, 128.7, 129.3, 135.7, 174.5, 177.0

HRMS: calcd for C₁₅H₁₉NO₃, (M+H)⁺262.1438, found 262.1433

### Example B2

### Compound B2 was Prepared According to General Procedure I Described Above

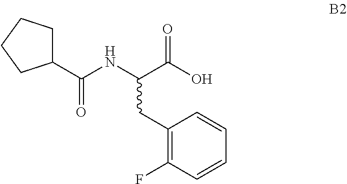 [SMILES: O=C(NC(Cc1ccccc1F)C(=O)O)C1CCCC1]

¹H NMR (500 MHz, CDCl₃) δ 1.54-1.83 (m, 8H), 2.52 (p, J=7.8, 15.8, 1H), 3.16 (dd, J=7.7, 14.2, 1H), 3.31 (dd, J=5.3, 14.2, 1H), 4.82 (q, J=7.5, 12.9, 1H), 6.05 (d, J=7.3, 1H), 7.02-7.11 (m, 2H), 7.19-7.25 (m, 2H)

¹³C NMR (125 MHz, CDCl₃): δ 25.77, 25.84, 29.98, 30.4, 30.8, 45.6, 52.9, 115.4 (d, J=22.3), 123.0 (d, J=15.5), 124.4 (d, J=3.4), 129.1 (d, J=8.2), 131.4 (d, J=4.4), 161.4 (d, J¹=243), 174.2, 177.3

HRMS: calcd for C₁₅H₁₈FNO₃, (M+H)⁺280.1343, found 280.1342

### Example B3

### Compound B3 was Prepared According to General Procedure I Described Above

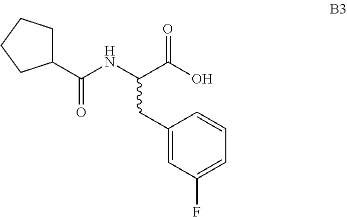 [SMILES: O=C(NC(Cc1cccc(F)c1)C(=O)O)C1CCCC1]

¹H NMR (500 MHz, CDCl₃) δ 1.55-1.57 (m, 2H), 1.68-1.73 (m, 4H), 1.82-1.84 (m, 2H), 2.5 (p, J=7.9, 15.7, 1H), 3.12 (dd, J=6.3, 14.1, 1H), 3.26 (dd, J=5.7, 14.1, 1H), 4.86 (q, J=6.0, 13.1, 1H), 5.97 (d, J=7.3, 1H), 6.86-6.89 (m, 1H), 6.94-6.98 (m, 2H), 7.24-7.29 (m, 1H)

¹³C NMR (125 MHz, CDCl₃): δ 25.8, 25.9, 30.1, 30.5, 36.9, 53.1, 114.2 (d, J=21.0), 116.3 (d, J=21.0), 125.0 (d, J=2.8), 130.1 (d, J=8.2), 138.3 (d, J=7.2), 162.8 (d, J¹=245), 174.1, 177.0

HRMS: calcd for C₁₅H₁₈FNO₃, (M+H)⁺280.1343, found 280.1337

### Example B4

### Compound B4 was Prepared According to General Procedure I Described Above

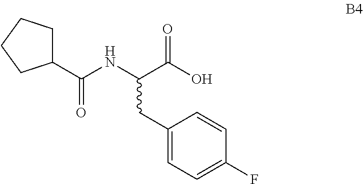 [SMILES: O=C(NC(Cc1ccc(F)cc1)C(=O)O)C1CCCC1]

¹H NMR (500 MHz, CDCl₃) δ 1.70-1.71 (m, 2H), 1.72-1.73 (m, 4H), 1.82-1.84 (m, 2H), 2.53 (q, J=8.0, 15.7, 1H), 3.10 (dd, J=6.2, 14.2, 1H), 3.23 (dd, J=5.7, 14.2, 1H), 4.84 (q, J=6.0, 13.3, 1H), 5.95 (d, J=7.4, 1H), 6.97-7.00 (m, 2H), 7.11-7.14 (m, 2H)

¹³C NMR (125 MHz, CDCl₃): δ 25.8, 25.9, 30.1, 30.5, 36.4, 45.6, 53.2, 115.5 (d, J=21.3), 130.9 (d, J=8.0), 131.4 (d, J=3.3), 162.1 (d, J¹=244), 174.3, 177.0

HRMS: calcd for C₁₅H₁₈FNO₃, (M+H)⁺280.1343, found 280.1347

### Example C1

### Compound Cl was Prepared According to General Procedure I Described Above

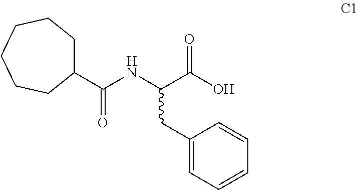 [SMILES: O=C(NC(Cc1ccccc1)C(=O)O)C1CCCCCC1]

¹H NMR (500 MHz, CDCl₃) δ 1.39-1.44 (m, 2H), 1.48-1.61 (m, 6H), 1.68-1.84 (m, 4H), 2.23 (septet, J=5.9, 10.0, 14.0, 1H), 3.12 (dd, J=6.5, 14.1, 1H), 3.25 (dd, J=5.6, 14.1, 1H), 4.85 (q, J=6.3, 13.1, 1H), 5.90 (d, J=7.4, 1H), 7.15-7.17 (m, 2H), 7.27-7.32 (m, 2H)

¹³C NMR (125 MHz, CDCl₃): δ 26.4, 26.5, 28.0, 28.1, 31.2, 31.5, 37.2, 47.1, 53.1, 127.2, 128.7, 129.4, 135.7, 174.6, 178.0

HRMS: calcd for C₁₇H₂₃NO₃, (M+H)⁺290.1751, found 290.1753

### Example C2

### Compound C2 was Prepared According to General Procedure I Described Above

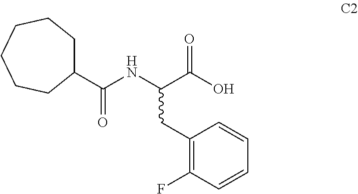 [SMILES: O=C(NC(Cc1ccccc1F)C(=O)O)C1CCCCCC1]

¹H NMR (500 MHz, CDCl₃) δ 1.40-1.44 (m, 2H), 1.49-1.58 (m, 6H), 1.68-1.80 (m, 4H), 2.23 (p, J=5.9, 10.0, 1H), 3.15 (dd, J=7.7, 14.2, 1H), 3.30 (dd, J=5.3, 14.2, 1H), 4.81 (q, J=1.8, 7.2, 1H), 6.03 (d, J=7.5, 1H), 7.02-7.10 (m, 2H), 7.19-7.25 (m, 2H)

¹³C NMR (125 MHz, CDCl₃): δ 26.4, 28.0, 28.1, 30.8, 31.1, 31.4, 47.1, 52.7, 115.4 (d, J=22.3), 123.0 (d, J=15.5), 124.4 (d, J=3.5), 129.1 (d, J=8.2), 131.7 (d, J=4.3), 161.4 (d, J¹=243), 174.4, 178.2

HRMS: calcd for C₁₇H₂₂FNO₃, (M+H)⁺308.1656, found 308.1664

### Example C3

### Compound C3 was Prepared According to General Procedure I Described Above

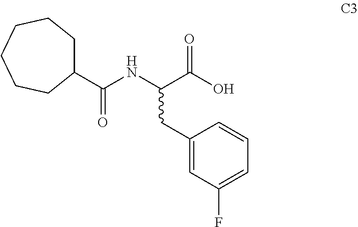 [SMILES: O=C(NC(Cc1cccc(F)c1)C(=O)O)C1CCCCCC1]

¹H NMR (500 MHz, CDCl₃) δ 1.40-1.45 (m, 2H), 1.50-1.61 (m, 6H), 1.70-1.75 (m, 2H), 1.81-1.84 (m, 2H), 2.26 (septet, J=5.9, 9.9, 14.0, 1H), 3.12 (dd, J=6.3, 14.1, 1H), 3.26 (dd, J=5.7, 14.1, 1H), 4.85 (q, J=6.0, 12.2, 1H), 5.97 (d, J=7.4, 1H), 6.86-6.89 (m, 1H), 6.93-6.97 (m, 2H)

¹³C NMR (125 MHz, CDCl₃): δ 26.4, 26.5, 28.0, 28.1, 31.2, 31.6, 37.0, 47.1, 52.9, 114.2 (d, J=20.8), 116.4 (d, J=21.2), 125.1 (d, J=2.6), 130.1 (d, J=8.2), 138.3 (d, J=7.2), 162.8 (d, J¹=245), 174.3, 178.0

HRMS: calcd for C₁₇H₂₂FNO₃, (M+H)⁺308.1656, found 308.1658

### Example C4

### Compound C4 was Prepared According to General Procedure I Described Above

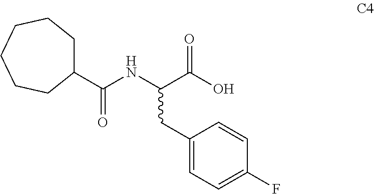 [SMILES: O=C(NC(Cc1ccc(F)cc1)C(=O)O)C1CCCCCC1]

¹H NMR (500 MHz, CDCl₃) δ 1.41-1.45 (m, 2H), 1.50-1.62 (m, 6H), 1.70-1.75 (m, 2H), 1.78-1.83 (m, 2H), 2.24 (septet, J=4.2, 5.9, 10.0, 1H), 3.10 (dd, J=6.4, 14.3, 1H), 3.23 (dd, J=5.7, 14.2, 1H), 4.82 (q, J=6.2, 13.2, 1H), 5.86 (d, J=7.3, 1H), 6.98-7.01 (m, 2H), 7.11-7.14 (m, 2H)

¹³C NMR (125 MHz, CDCl₃): δ 26.42, 26.44, 28.0, 28.1, 31.2, 31.6, 36.4, 47.1, 53.1, 115.5 (d, J=21.0), 130.9 (d, J=8.0), 131.4 (d, J=3.3), 162.1 (d, J¹=244), 174.1, 177.9

HRMS: calcd for C₁₇H₂₂FNO₃, (M+H)⁺308.1656, found 308.1649

The following additional compound examples are described herein:

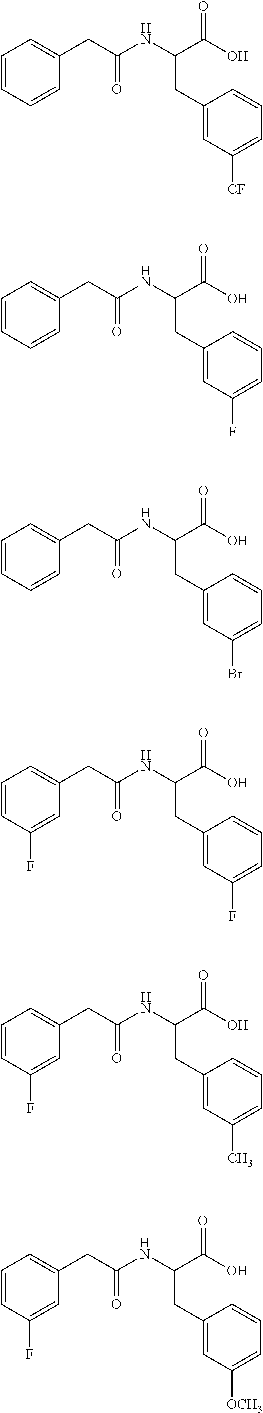

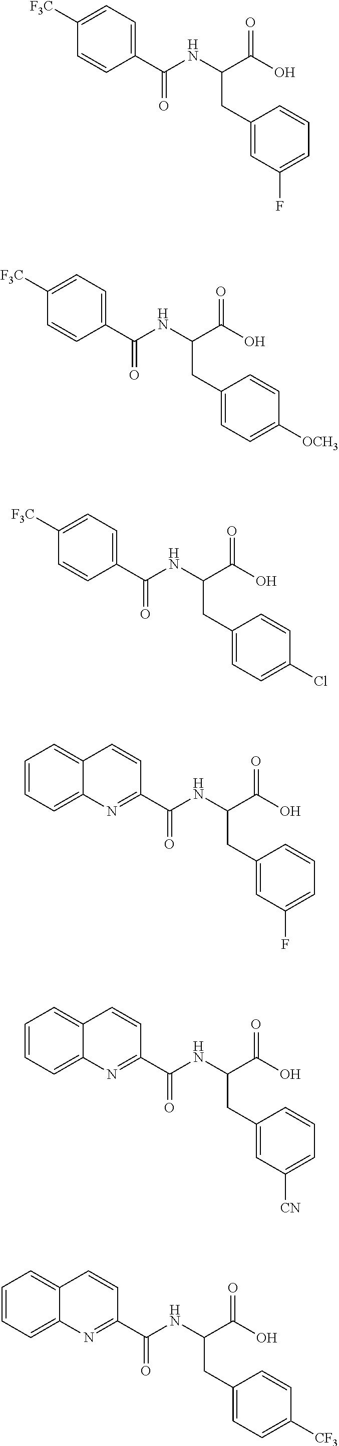 [SMILES: O=C(NC(Cc1cccc(F)c1)C(=O)O)c1ccc(C(F)(F)F)cc1 ; COc1ccc(CC(NC(=O)c2ccc(C(F)(F)F)cc2)C(=O)O)cc1 ; O=C(NC(Cc1ccc(Cl)cc1)C(=O)O)c1ccc(C(F)(F)F)cc1 ; O=C(NC(Cc1cccc(F)c1)C(=O)O)c1ccc2ccccc2n1 ; N#Cc1cccc(CC(NC(=O)c2ccc3ccccc3n2)C(=O)O)c1 ; O=C(NC(Cc1ccc(C(F)(F)F)cc1)C(=O)O)c1ccc2ccccc2n1]

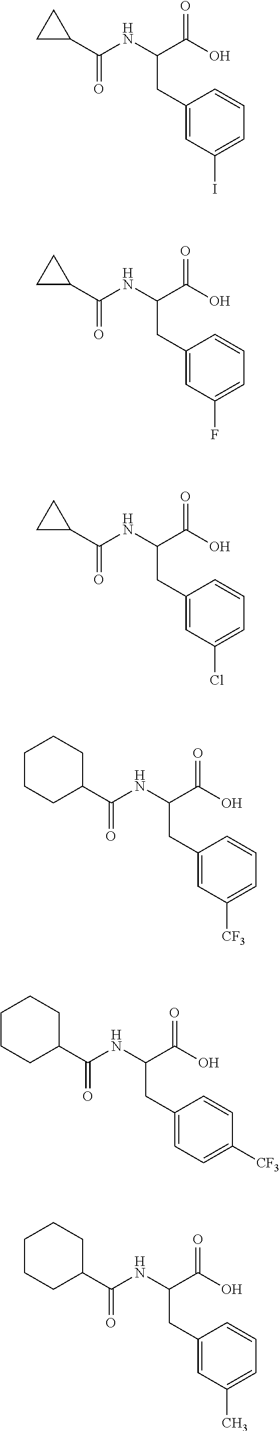 [SMILES: O=C(NC(Cc1cccc(I)c1)C(=O)O)C1CC1 ; O=C(NC(Cc1cccc(F)c1)C(=O)O)C1CC1 ; O=C(NC(Cc1cccc(Cl)c1)C(=O)O)C1CC1 ; O=C(NC(Cc1cccc(C(F)(F)F)c1)C(=O)O)C1CCCCC1 ; O=C(NC(Cc1ccc(C(F)(F)F)cc1)C(=O)O)C1CCCCC1 ; Cc1cccc(CC(NC(=O)C2CCCCC2)C(=O)O)c1]

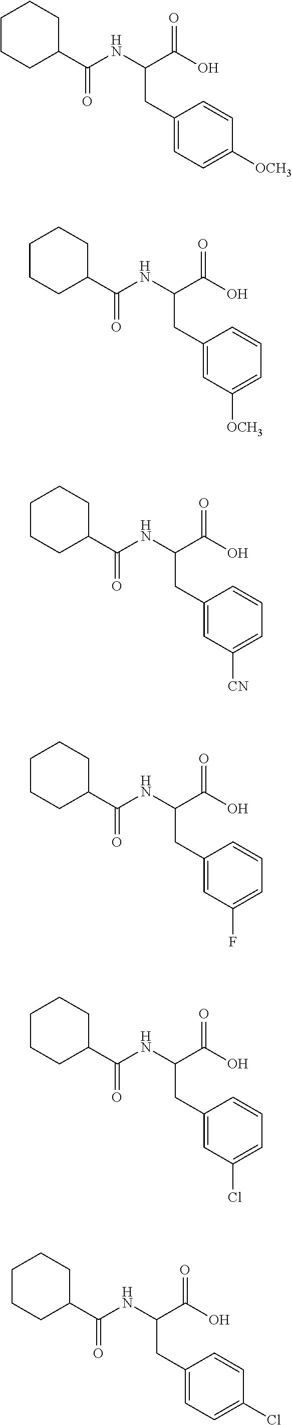 [SMILES: COc1ccc(CC(NC(=O)C2CCCCC2)C(=O)O)cc1 ; COc1cccc(CC(NC(=O)C2CCCCC2)C(=O)O)c1 ; N#Cc1cccc(CC(NC(=O)C2CCCCC2)C(=O)O)c1 ; O=C(NC(Cc1cccc(F)c1)C(=O)O)C1CCCCC1 ; O=C(NC(Cc1cccc(Cl)c1)C(=O)O)C1CCCCC1 ; O=C(NC(Cc1ccc(Cl)cc1)C(=O)O)C1CCCCC1]

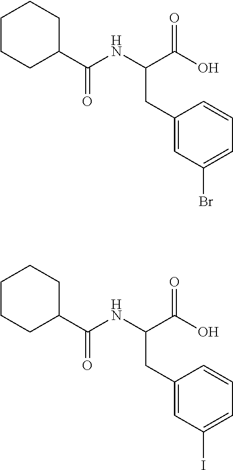 [SMILES: O=C(NC(Cc1cccc(Br)c1)C(=O)O)C1CCCCC1 ; O=C(NC(Cc1cccc(I)c1)C(=O)O)C1CCCCC1]

### Glucose Stimulated Insulin Secretion Assay

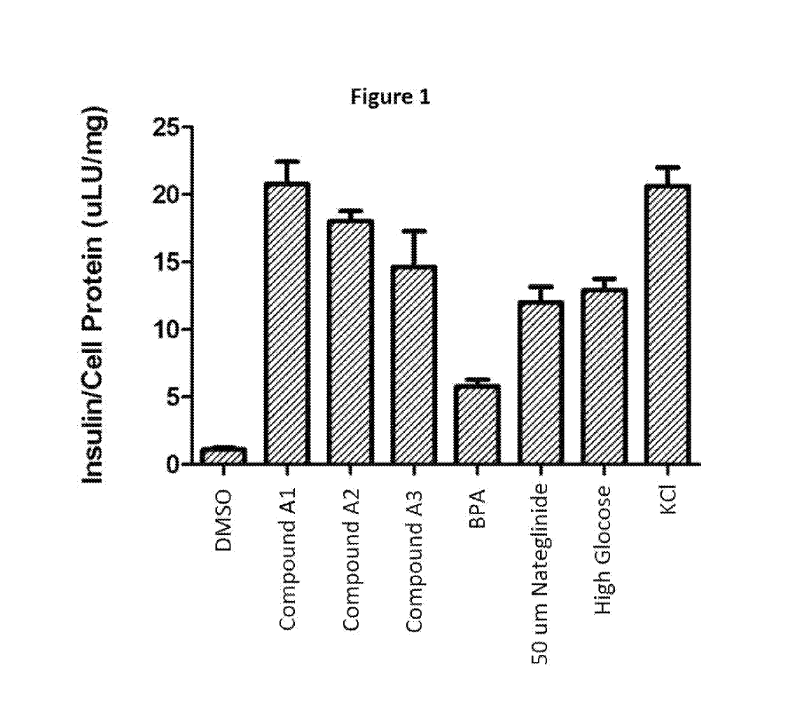

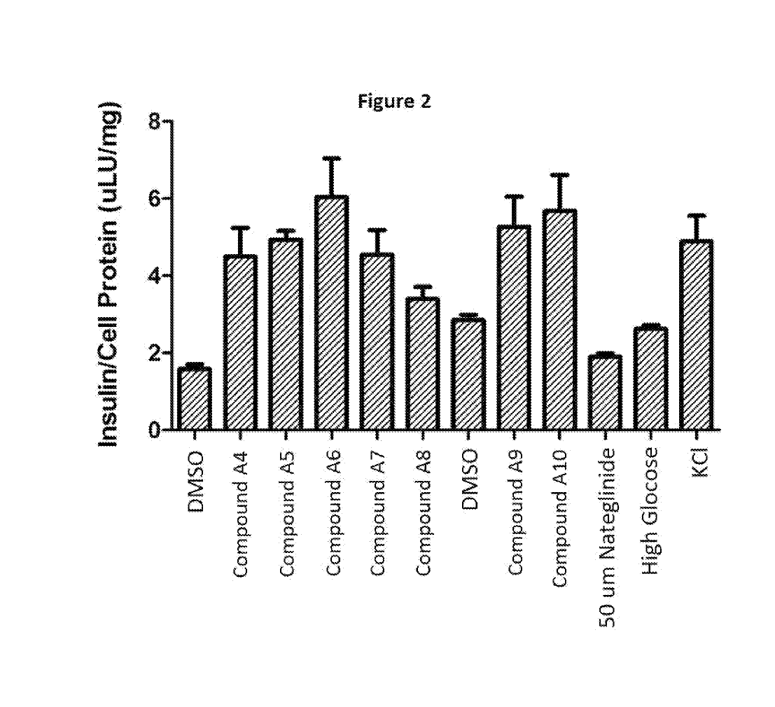

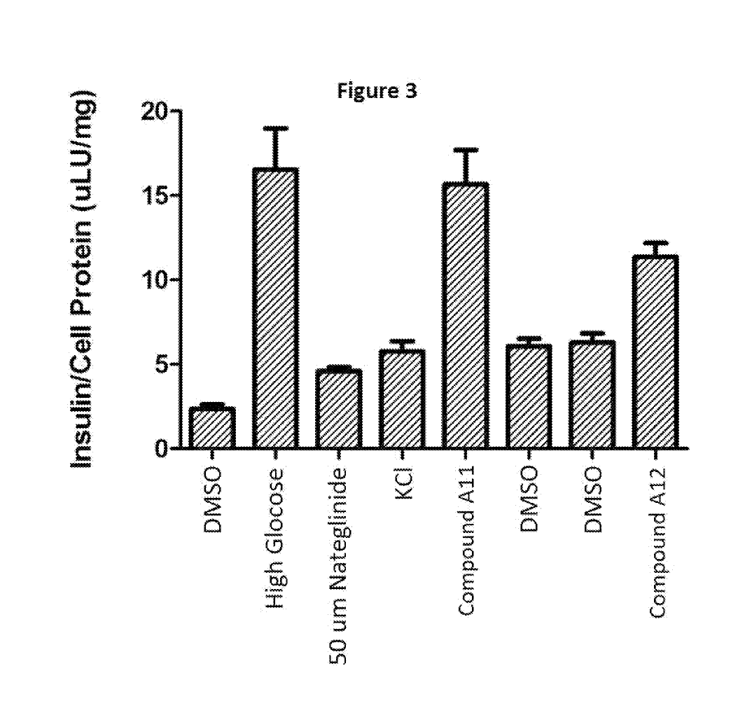

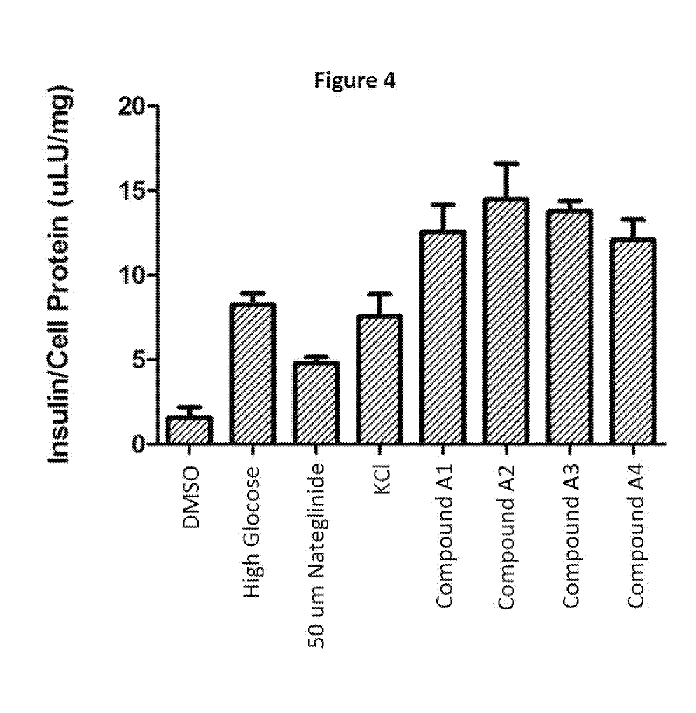

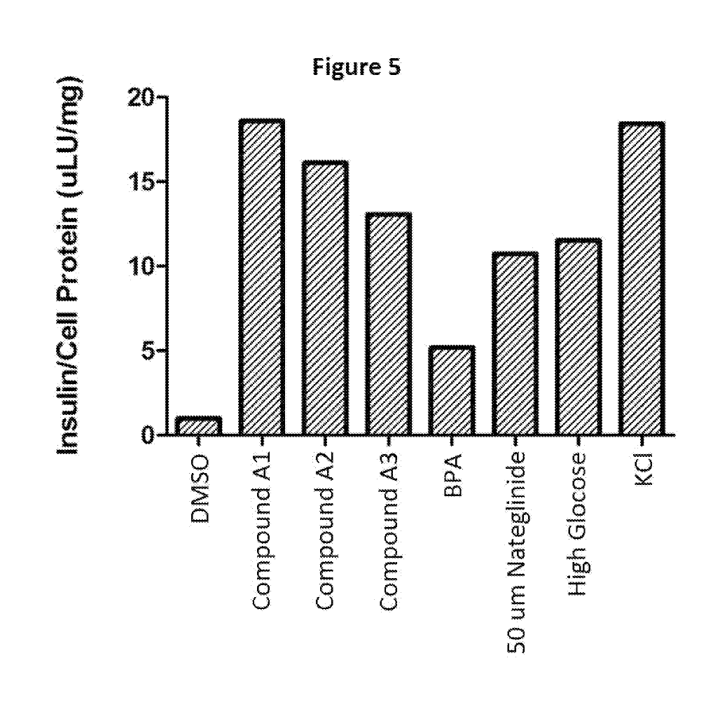

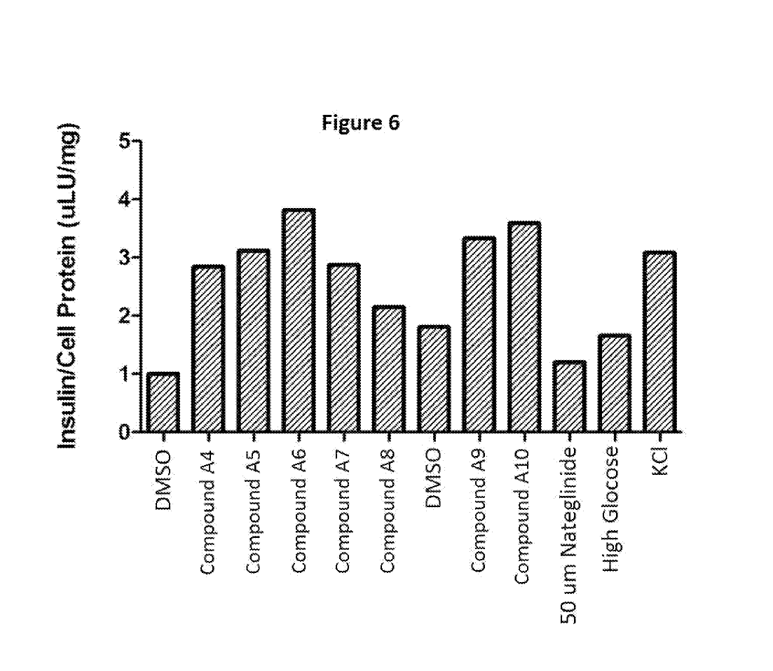

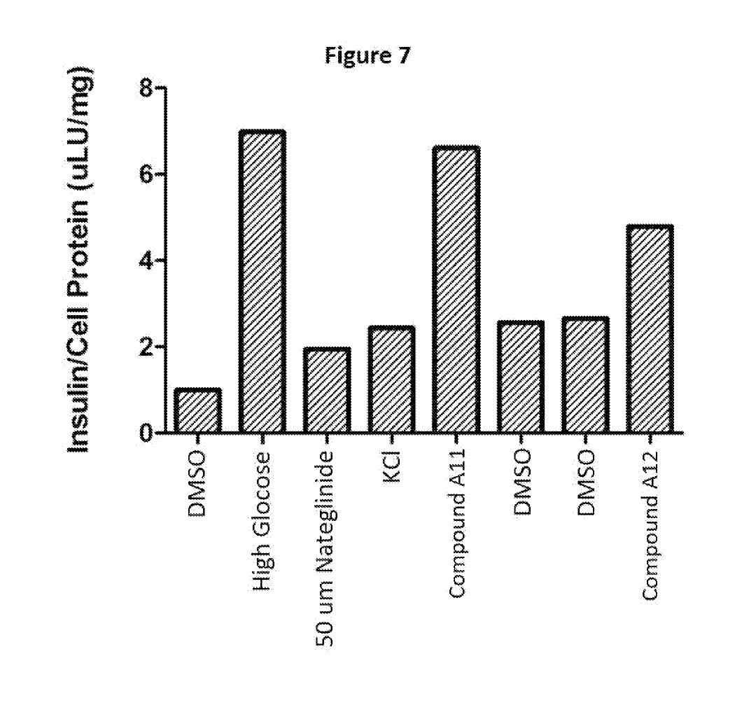

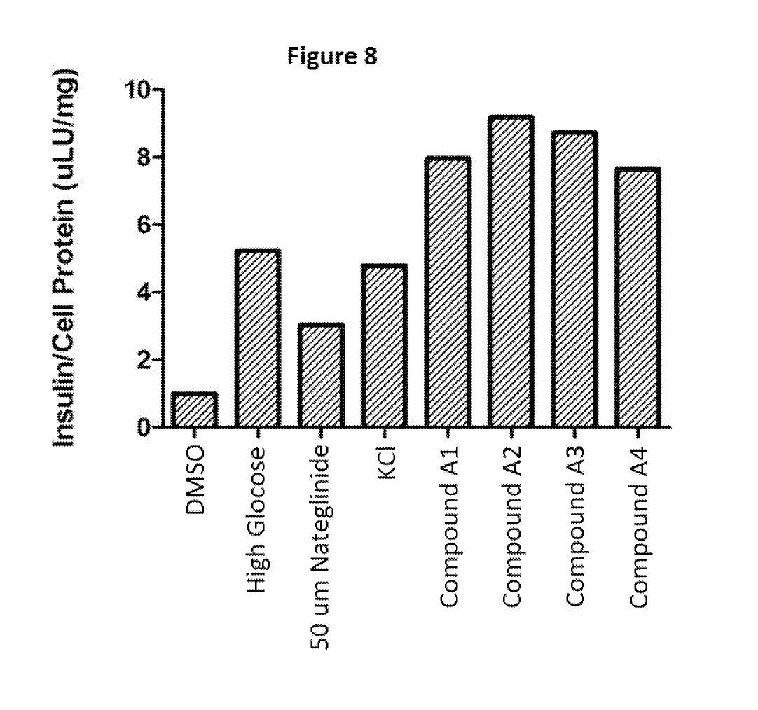

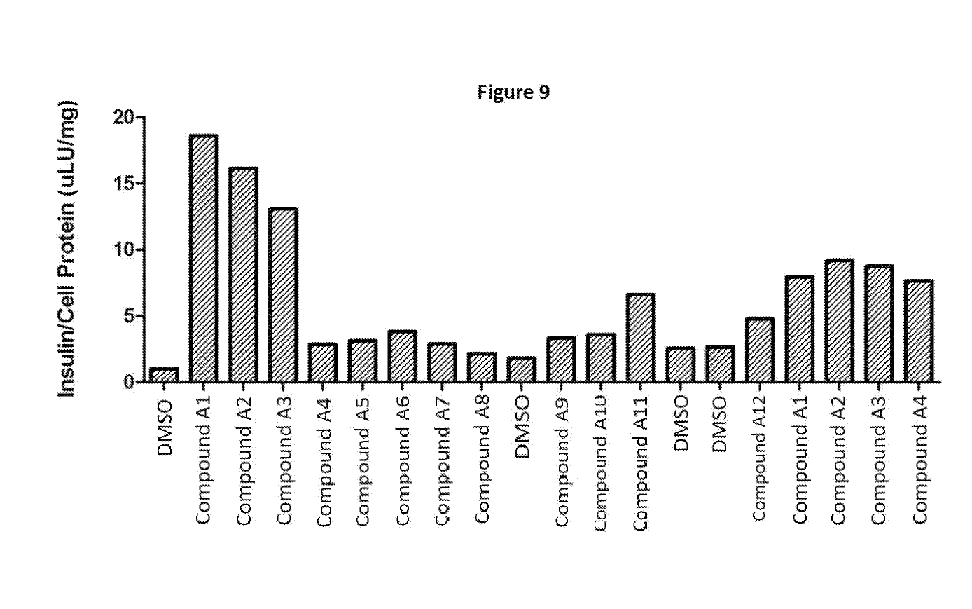

INS-1 cells (832/13) were cultured in RPMI-1640 containing 11.1 mM glucose supplemented with 10% fetal bovine serum, 100 U/ml penicillin, 100 μg/ml streptomycin, 10 mM HEPES, 2 mM L-glutamine, 1 mM sodium pyruvate, and 50 μM β-mercaptoethanol and then seeded in twelve well plates until they reached confluency. To assess insulin secretion, cells were pre-incubated in SAB solution with 2.5 mM glucose for two hours and then switched to SAB solution containing either 2.5 mM glucose and solvent DMSO control, 250 uM of each test compound, nateglinide 50 uM, or KCL 30 mM or media containing 11 mM glucose (high glucose or HG) for an additional two hours. Media was collected for insulin determination by radioimmunoassay, and the total amount of insulin secretion was normalized to protein content of the cells. Results are shown in FIGS. 1-9.

The following publications, and each of the additional publications cited herein are incorporated herein by reference:

1. Scott, W. L.; O′Donnell, M. J. Distributed Drug Discovery, Part 1: Linking Academics and Combinatorial Chemistry to Find Drugs for Developing World Diseases. J. Comb. Chem. 2009, 11, 3-13.

2. Scott, W. L.; Alsina, J.; Audu, C. O.; Dage, J. L.; Babaev, E.; Cook, L.; Goodwin, L. A.; Martynow, J. G.; Matosiuk, D.; Royo, M.; Smith, J. G.; Strong, A. T.; Wickizer, K.; Woerly, E. M.; Zhou, Z.; O′Donnell, M. J. Distributed Drug Discovery, Part 2: Global Rehearsal of Alkylating Agents for the Synthesis of Resin-Bound Unnatural Amino Acids and Virtual D³ Catalog Construction. J. Comb. Chem. 2009, 11, 14-33.

3. Scott, W. L.; Audu, C. O.; Dage, J. L.; Goodwin, L. A.; Martynow, J. G.; Platt, L. K; Smith, J. G.; Strong, A. T.; Wickizer, K.; Woerly, E. M.; O′Donnell, M. J. Distributed Drug Discovery, Part 3: Using D³ Methodology to Synthesize Analogs of an Anti-Melanoma Compound. J. Comb. Chem. 2009, 11, 34-43.

4. J. Med. Chem. 1988, 31, 2092-2097.

5. J. Med. Chem. 1989, 32, 1436-1441.

What is claimed is:

1. A compound of the formula I 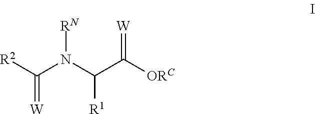
or a pharmaceutically acceptable salt thereof, wherein:
W is independently O or S;
R^{N} is H, or a amide prodrug forming group;
R^{C} is H, or a carboxylic acid prodrug forming group;
R¹ is arylalkyl or heteroarylalkyl, each of which is optionally substituted; and
R² is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted;
wherein the compound is not of the formula 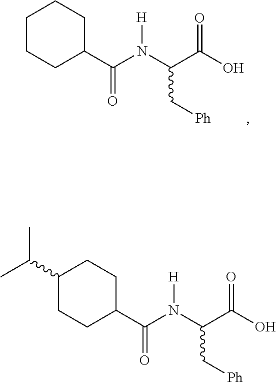 [SMILES: O=C(NC(Cc1ccccc1)C(=O)O)C1CCCCC1 ; CC(C)C1CCC(C(=O)NC(Cc2ccccc2)C(=O)O)CC1]
or a pharmaceutically acceptable salt thereof.

2. The compound of claim 1, wherein R² is selected from cyclopentyl, cyclohexyl, and cycloheptyl, each of which is optionally substituted, or a pharmaceutically acceptable salt thereof.

3. The compound of claim 1, wherein R² is optionally substituted cyclopentyl, or a pharmaceutically acceptable salt thereof.

4. The compound of claim 1, wherein R² is cyclopentyl substituted with at least one C₁-C₆ alkyl, or a pharmaceutically acceptable salt thereof.

5. The compound of claim 1, wherein R² is optionally substituted cyclohexyl, or a pharmaceutically acceptable salt thereof.

6. The compound of claim 1, wherein R² is cyclohexyl substituted with at least one C₁-C₆ alkyl, or a pharmaceutically acceptable salt thereof.

7. The compound of claim 1, wherein R² is optionally substituted cycloheptyl, or a pharmaceutically acceptable salt thereof.

8. The compound of claim 1, wherein R² is cycloheptyl substituted with at least one C₁-C₆ alkyl, or a pharmaceutically acceptable salt thereof.

9. The compound of claim 1, wherein W is O, or a pharmaceutically acceptable salt thereof.

10. The compound of claim 1, wherein R^{N} is H, or a pharmaceutically acceptable salt thereof.

11. The compound of claim 1, wherein R^{C} is H, or a pharmaceutically acceptable salt thereof.

12. The compound of claim 1, wherein R¹ is optionally substituted arylalkyl, or a pharmaceutically acceptable salt thereof.

13. The compound of claim 1, wherein R¹ is optionally substituted benzyl, or a pharmaceutically acceptable salt thereof.

14. The compound of claim 1, wherein R¹ is benzyl substituted with one or more halogen atom, or a pharmaceutically acceptable salt thereof.

15. The compound of claim 14, wherein the halogen atom is fluorine, or a pharmaceutically acceptable salt thereof.

16. The compound of claim 14, wherein the halogen atom is chlorine or bromine, or a pharmaceutically acceptable salt thereof.

17. The compound of claim 1, selected from the group consisting of 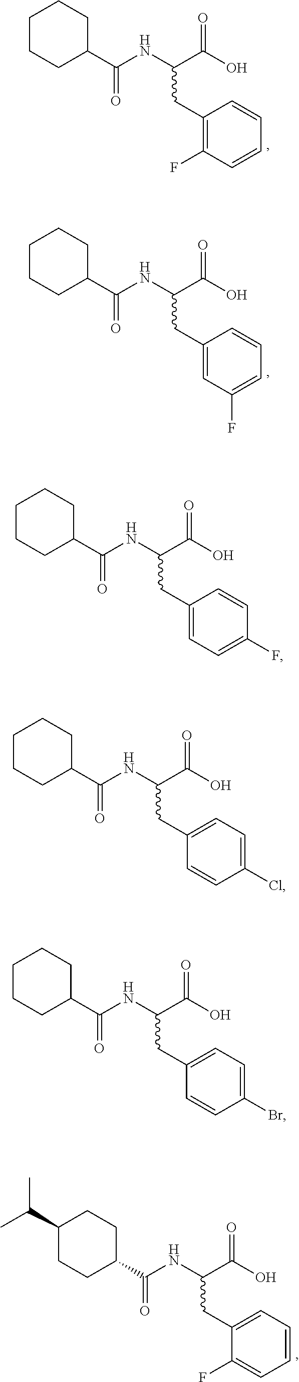 [SMILES: O=C(NC(Cc1ccccc1F)C(=O)O)C1CCCCC1 ; O=C(NC(Cc1cccc(F)c1)C(=O)O)C1CCCCC1 ; O=C(NC(Cc1ccc(F)cc1)C(=O)O)C1CCCCC1 ; O=C(NC(Cc1ccc(Cl)cc1)C(=O)O)C1CCCCC1 ; O=C(NC(Cc1ccc(Br)cc1)C(=O)O)C1CCCCC1 ; CC(C)[C@H]1CC[C@H](C(=O)NC(Cc2ccccc2F)C(=O)O)CC1] 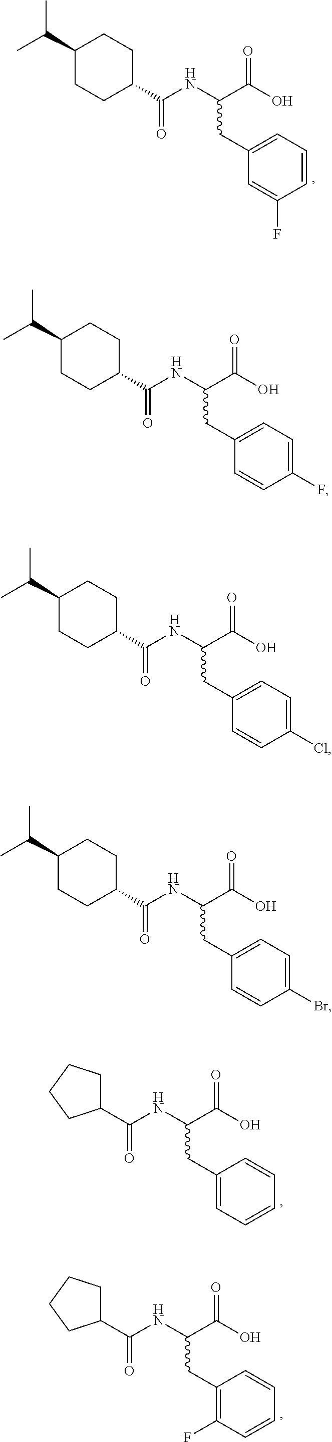 [SMILES: CC(C)[C@H]1CC[C@H](C(=O)NC(Cc2cccc(F)c2)C(=O)O)CC1 ; CC(C)[C@H]1CC[C@H](C(=O)NC(Cc2ccc(F)cc2)C(=O)O)CC1 ; CC(C)[C@H]1CC[C@H](C(=O)NC(Cc2ccc(Cl)cc2)C(=O)O)CC1 ; CC(C)[C@H]1CC[C@H](C(=O)NC(Cc2ccc(Br)cc2)C(=O)O)CC1 ; O=C(NC(Cc1ccccc1)C(=O)O)C1CCCC1 ; O=C(NC(Cc1ccccc1F)C(=O)O)C1CCCC1] 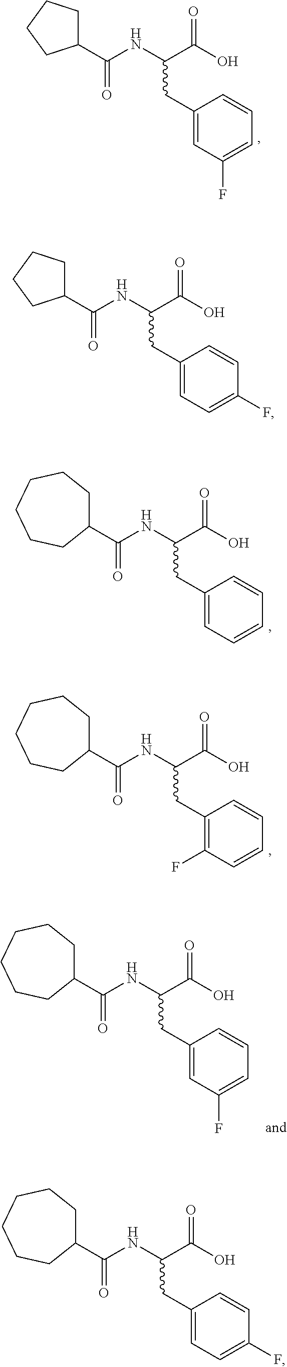 [SMILES: O=C(NC(Cc1cccc(F)c1)C(=O)O)C1CCCC1 ; O=C(NC(Cc1ccc(F)cc1)C(=O)O)C1CCCC1 ; O=C(NC(Cc1ccccc1)C(=O)O)C1CCCCCC1 ; O=C(NC(Cc1ccccc1F)C(=O)O)C1CCCCCC1 ; O=C(NC(Cc1cccc(F)c1)C(=O)O)C1CCCCCC1 ; O=C(NC(Cc1ccc(F)cc1)C(=O)O)C1CCCCCC1]
or a pharmaceutically acceptable salt thereof.

18. A pharmaceutical composition comprising a compound of claim 1, and one or more carriers, diluents, or excipients.

19. A method for treating a disease responsive to increased insulin production in a mammal in need of such treatment, the method comprising the step of administering to the mammal a therapeutically effective amount of a compound of claim 1.

20. The method of claim 19, wherein the disease responsive to increased insulin production is type 2 diabetes.
